(19) United States
(12) Patent Application Publication
(10) Pub. No.: US 2010/0249438 A1
(43) Pub. Date: Sep. 30, 2010

(54) PREPARATION OF ESCITALOPRAM

(76) Inventors: Vijaya Bhaskar Bolugoddu, Hyderabad (IN); Chandrasekhar Ravi Ram Elati, Hyderabad (IN); Sasi Kala Venkata Annapurna Cheemalapati, Hyderabad (IN); Rajagopal Penumandla, Warangal (IN); Arulnathan Prabakar Christy, Trichy (IN); Lakshmi Vara Prasad Sree Naga Venkata Vakamudi, Krishna (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/749,569

(22) Filed: Mar. 30, 2010

Related U.S. Application Data
(60) Provisional application No. 61/186,596, filed on Jun. 12, 2009.

(30) Foreign Application Priority Data
Mar. 30, 2009 (IN) 705/CHE/2009

(51) Int. Cl. C07D 307/79

(52) U.S. Cl. 549/469

(57) ABSTRACT

A substantially pure (S)-4-[4-(dimethylamino)-1-(4′-fluorophenyl)-1-hydroxybutyl]-3-(hydroxymethyl)benzonitrile intermediate for preparing escitalopram is prepared by:

a) combining racemic (±)-4-[4-(dimethylamino)-1-(4′-fluorophenyl)-1-hydroxybutyl]-3-(hydroxymethyl)benzonitrile with (−)-di-p-toluoyltartaric acid, in a solvent;

b) separating a solid phase comprising a salt of (−)-di-p-toluyltartaric acid with (R)-4-[4-(dimethylamino)-1-(4′-fluorophenyl)-1-hydroxybutyl]-3-(hydroxymethyl)benzonitrile enantiomer, and a liquid phase comprising a salt of (−)-di-p-toluoyltartaric acid with (S)-4-[4-(dimethylamino)-1-(4′-fluorophenyl)-1-hydroxy-butyl]-3-(hydroxymethyl)benzonitrile enantiomer;

c) reacting the liquid phase with a base and isolating enantiomerically enriched (S)-4-[4-(dimethylamino)-1-(4′-fluorophenyl)-1-hydroxybutyl]-3-(hydroxymethyl)benzonitrile enantiomer;

d) combining enantiomerically enriched (S)-enantiomer obtained in c) with (+)-di-p-toluoyltartaric acid, in a solvent; and

e) reacting a precipitate from d) with a base.

Aspects of the present application relate to processes for the preparation of escitalopram and salts thereof.

Escitalopram is the S-enantiomer of racemic citalopram, which is a bicyclic phthalane derivative. The structure of escitalopram can be represented as Formula I.

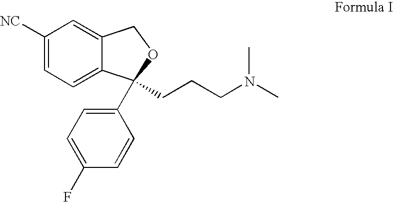

Escitalopram is a selective serotonin reuptake inhibitor useful in the treatment of depression and is commercially available in the USA, in the form of its oxalate salt, as the active ingredient in LEXAPRO™ products. Escitalopram oxalate has a chemical name S-(+)-1-[3-(dimethylamino)propyl]-1-(p-fluorophenyl)-5-phthalancarbonitrile oxalate.

U.S. Pat. No. RE34712 discloses a process for the preparation of escitalopram. The process involves the resolution of 4-[4-(dimethylamino)-1-(4′-fluorophenyl)-1-hydroxybutyl]-3-(hydroxymethyl)benzonitrile, using chiral di-p-toluoyltaratric acid, and cyclization of the desired isomer to obtain escitalopram.

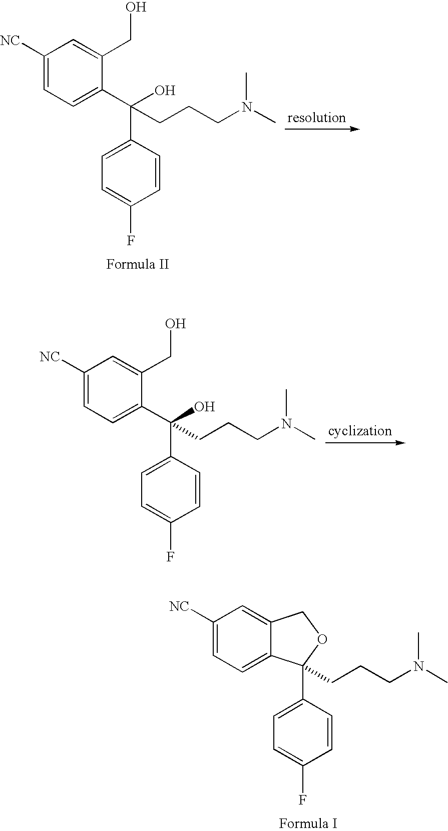

Resolution of the intermediate of Formula II (the “diol” intermediate) using the disclosed processes proved to be not fully satisfactory, largely with respect to low product yield.

Hence, there remains a need for improved processes to prepare (S)-4-[4-(dimethylamino)-1-(4′-fluorophenyl)-1-hydroxybutyl]-3-(hydroxymethyl)benzonitrile, as well as escitalopram, to improve optical purity and overall yield.

### SUMMARY

Aspects of the invention provide processes for the preparation of escitalopram and its pharmaceutically acceptable salts. In particular aspects, the invention provides processes for the enantiomeric enrichment of racemic 4-[4-(dimethylamino)-1-(4′-fluorophenyl)-1-hydroxybutyl]-3-(hydroxymethyl)-benzonitrile of Formula II, hereinafter designated as the “diol” intermediate, and processes to obtain enantiomerically pure diol intermediate from enantiomerically enriched diol intermediate.

In an aspect, there is provided a process for preparing escitalopram, or a salt thereof, comprising:

a) combining racemic (±)-4-[4-(dimethylamino)-1-(4′-fluorophenyl)-1-hydroxybutyl]-3-(hydroxymethyl)benzonitrile with (−)-di-p-toluoyltartaric acid, in a solvent;

b) separating a solid phase comprising a salt of (−)-di-p-toluyltartaric acid with (R)-4-[4-(dimethylamino)-1-(4′-fluorophenyl)-1-hydroxybutyl]-3-(hydroxymethyl)benzonitrile enantiomer, and a liquid phase comprising a salt of (−)-di-p-toluoyltartaric acid with (S)-4-[4-(dimethylamino)-1-(4′-fluorophenyl)-1-hydroxy-butyl]-3-(hydroxymethyl)benzonitrile enantiomer;

c) reacting the liquid phase with a base and isolating enantiomerically enriched (S)-4-[4-(dimethylamino)-1-(4′-fluorophenyl)-1-hydroxybutyl]-3-(hydroxymethyl)benzonitrile enantiomer;

d) combining enantiomerically enriched (S)-enantiomer obtained in c) with (+)-di-p-toluoyltartaric acid, in a solvent; and

e) reacting a precipitate from d) with a base to produce (S)-4-[4-(dimethylamino)-1-(4′-fluorophenyl)-1-hydroxybutyl]-3-(hydroxymethyl)benzonitrile.

In an aspect, there is provided a process for preparing escitalopram, or a salt thereof, comprising:

a) combining racemic (±)-4-[4-(dimethylamino)-1-(4′-fluorophenyl)-1-hydroxybutyl]-3-(hydroxymethyl)benzonitrile with (−)-di-p-toluoyltartaric acid, in a solvent comprising an alcohol, a ketone, an ether, a hydrocarbon, a nitrile, or any mixtures thereof;

b) separating a solid phase comprising a salt of (−)-di-p-toluyltartaric acid with (R)-4-[4-(dimethylamino)-1-(4′-fluorophenyl)-1-hydroxybutyl]-3-(hydroxymethyl)-benzonitrile enantiomer, and a liquid phase comprising a salt of (−)-di-p-toluoyltartaric acid with (S)-4-[4-(dimethylamino)-1-(4′-fluorophenyl)-1-hydroxy-butyl]-3-(hydroxymethyl)benzonitrile enantiomer;

c) reacting the liquid phase with a base and isolating enantiomerically enriched (S)-4-[4-(dimethylamino)-1-(4′-fluorophenyl)-1-hydroxybutyl]-3-(hydroxymethyl)benzonitrile enantiomer;

d) combining enantiomerically enriched (S)-enantiomer obtained in c) with (+)-di-p-toluoyltartaric acid, in a solvent comprising an alcohol, a ketone, an ether, a hydrocarbon, a nitrile, or any mixtures thereof;

e) reacting a precipitate from d) with a base to produce substantially pure (S)-4-[4-(dimethylamino)-1-(4′-fluorophenyl)-1-hydroxybutyl]-3-(hydroxymethyl)-benzonitrile; and

f) converting substantially pure (S)-4-[4-(dimethylamino)-1-(4′-fluorophenyl)-1-hydroxybutyl]-3-(hydroxymethyl)benzonitrile into escitalopram.

### DETAILED DESCRIPTION

Aspects of the present application provide processes to prepare a substantially pure S-enantiomer of the diol intermediate, embodiments comprising:

(1) combining the racemic diol intermediate with (−)-di-p-toluoyltartaric acid in a solvent;

(2) precipitating a solid phase comprising predominantly a salt of (−)-di-p-toluyltartaric acid with an (R)-enantiomer of the diol intermediate, and a liquid phase comprising predominantly a salt of (−)-di-p-toluoyltartaric acid with an (S)-enantiomer of the diol intermediate;

(3) separating the solid phase;

(4) reacting the liquid phase with base and isolating the diol intermediate, enantiomerically enriched with (S)-enantiomer;

(5) combining the enriched diol intermediate enantiomer obtained in (4) with (+)-di-p-toluoyltartaric acid in a solvent;

(6) precipitating a diastereomeric salt of (+)-di-p-toluoyltartaric acid with the (S)-enantiomer of the diol intermediate; and

(7) isolating a substantially pure (S)-enantiomer of the diol intermediate by reacting the precipitate of (6) with a base.

As set forth in (1) above, a racemic diol intermediate and an appropriate quantity of (−)-di-p-toluoyltartaric acid are combined in a suitable solvent.

The term “racemic,” as used herein, refers to a mixture of a (R) and (S) enantiomers in any proportions. For example, the racemic diol intermediate used in the step (1) may be present in the form of a racemate wherein the ratio of R and S enantiomer is about 1:1, or one of the enantiomers may be slightly in excess.

The amount of optically active (−)-di-p-toluoyltartaric acid used may be less than, equal to, or greater than the molar equivalent of racemic diol intermediate. In embodiments, the amount of acid is used in ratios from about 0.15 to about 0.95 moles, per mole of diol intermediate.

The diol intermediate and (−)-di-p-toluoyltartaric acid may be combined in a suitable solvent or mixture of solvents. Suitable solvents include, but are not limited to: alcohols such as methanol, ethanol, isopropyl alcohol, and n-propanol; halogenated hydrocarbons such as dichloromethane, 1,2-dichloroethane, chloroform, and carbon tetrachloride; ketones such as acetone, ethyl methyl ketone, and methyl isobutyl ketone; esters such as ethyl acetate, n-propyl acetate, n-butyl acetate, and t-butyl acetate; ethers such as diethyl ether, dimethyl ether, diisopropyl ether, methyl t-butyl ether, tetrahydrofuran, and 1,4-dioxane; hydrocarbons such as toluene, xylene, n-heptane, cyclohexane, and n-hexane; nitriles such as acetonitrile and propionitrile; and any mixtures thereof.

The mixture may be stirred at suitable temperatures, such as about 25° C. to about the reflux temperature of the solvent or mixture of solvents used. The formed solid may be isolated using any solid separation techniques, as are well known to the person skilled in the art, such as filtration, decantation, or centrifugation. The solid comprises mostly the salt of (−)-di-p-toluoyltartaric acid with the (R)-enantiomer of the diol intermediate.

The liquid phase, comprising a salt of (−)-di-p-toluoyltartaric acid with the diol intermediate, which is predominantly an (S)-enantiomer, may be reacted with a base to obtain the diol intermediate, enantiomerically enriched with (S)-enantiomer. The liquid phase may be evaporated and the resulting residue may be treated with a base in a solvent or mixture of solvents, or the liquid phase may be directly treated with a base to release the diol intermediate from the di-p-toluoyltartaric acid salt.

Non-limiting examples of suitable bases include: alkali metal or alkaline earth metal hydroxides such as sodium hydroxide, potassium hydroxide, magnesium hydroxide and calcium hydroxide; carbonates such as sodium carbonate and potassium carbonate; and amines such as triethylamine. Suitable solvents include, but are not limited to: water; lower alcohols such as methanol, ethanol, n-propanol, isopropanol, isobutanol, and t-butanol; esters such as ethyl acetate, isopropyl acetate, butyl acetate and isobutyl acetate; halogenated hydrocarbons such as dichloromethane, dichloroethane, and chloroform; ketones such as acetone, methyl ethyl ketone, and methyl isobutyl ketone; ethers such as tetrahydrofuran, diethyl ether, diisopropyl ether. and dioxane; and any mixtures thereof.

The (S)-enantiomer predominates in the isolated diol intermediate. For example, the diol intermediate which is enantiomerically enriched by the said process contains about 60-80%, or about 70%, by weight of S-enantiomer.

The enantiomerically enriched diol intermediate may be further reacted with an optically active acid such as (+)-di-p-toluoyltartaric acid, thereby further enhancing the enantiomeric purity to obtain a substantially pure (S)-diol intermediate. The term “substantially pure” means that the material has an enantiomeric excess of at least about 90%, or at least about 95%, or at least about 98%, by weight of the desired enantiomer.

In a typical process, the enantiomerically enriched diol intermediate may be reacted with (+)-di-p-toluoyltartaric acid in a suitable solvent.

The amount of (+)-di-p-toluoyltartaric acid used may be less than, equal to, or greater than the molar equivalent of diol intermediate. In embodiments, the amount of the acid is used in ratio from about 0.15 to about 0.95 moles, per mole of diol intermediate.

Suitable solvents include, but are not limited to: lower alcohols such as methanol, ethanol, n-propanol, isopropanol, isobutanol, and t-butanol; esters such as ethyl acetate, isopropyl acetate, butyl acetate, and isobutyl acetate; halogenated hydrocarbons such as dichloromethane, dichloroethane, and chloroform; ketones such as acetone, methyl ethyl ketone and methyl isobutyl ketone; ethers such as tetrahydrofuran, diethyl ether, diisopropyl ether, and dioxane; and any mixtures thereof.

The mixture may be stirred at suitable temperatures, such as about 25° C. to about the solvent reflux temperature. The formed solid may be isolated using techniques such as decantation, filtration, centrifugation, etc.

Optionally, the di-p-toluoyltartaric acid salt of the diol intermediate may be treated with a suitable solvent or mixture of solvents to enhance the purity. The treatment may involve recrystallization of salt from suitable solvent or it may involve washing of the salt using a suitable solvent.

Useful solvents include, without limitation: lower alcohols such as methanol, ethanol, n-propanol, isopropanol, isobutanol, and t-butanol; esters such as ethyl acetate, isopropyl acetate, butyl acetate, and isobutyl acetate; halogenated hydrocarbons such as dichloromethane, dichloroethane, and chloroform; ketones such as acetone, methyl ethyl ketone, and methyl isobutyl ketone; ethers such as tetrahydrofuran, diethyl ether, diisopropyl ether, and dioxane; and any mixtures thereof.

The obtained solid may be reacted with a suitable base, in the presence of a suitable solvent, to obtain a substantially pure (S)-enantiomer of diol intermediate.

Examples of suitable bases include, without limitation thereto, alkali metal or alkaline earth metal hydroxides such as sodium hydroxide and potassium hydroxide, magnesium hydroxide and calcium hydroxide, carbonates such as sodium carbonate and potassium carbonate, and amines such as triethylamine.

Suitable solvents include, without limitation: water; lower alcohols such as methanol, ethanol, n-propanol, isopropanol, isobutanol, and t-butanol; esters such as ethyl acetate, isopropyl acetate, butyl acetate, and isobutyl acetate; halogenated hydrocarbons such as dichloromethane, dichloroethane, and chloroform; ketones such as acetone, methyl ethyl ketone and methyl isobutyl ketone; ethers such as tetrahydrofuran, diethyl ether, diisopropyl ether, and dioxane; and any mixtures thereof.

The solvent used for the preparation of a salt of di-p-toluoyltartaric acid with the diol intermediate may affect the optical purity and the yield of the desired enantiomer. Particularly, it has been observed that the solvent can affect the yield.

In embodiments, the enantiomerically enriched diol intermediate may be reacted with (+)-di-p-toluoyltartaric acid in ethyl acetate, or mixtures of ethyl acetate and an alcohol. The resulting salt may be subsequently treated with a base to obtain substantially pure (S)-enantiomer of diol intermediate in high yield, such as about 80% to about 90%, and a chiral purity greater than about 98% by weight.

The (S)-enantiomer of the diol intermediate prepared according to the processes of the present application is of high enantiomeric purity, such as at least about 98% by weight, as determined using chiral high performance liquid chromatography (HPLC) and the yield of the desired isomer can be in the range of about 80% to about 90%.

The foregoing processes may further include the steps of recovering an undesired enantiomer of the diol intermediate, racemizing, and recycling the racemic mixture.

The processes disclosed above may also be used for making a substantially pure (R)-enantiomer of the diol intermediate, by using (+)-di-p-toluoyltartaric acid in the first step, along with corresponding modifications to the subsequent steps.

In embodiments, the present application provides processes for increasing the enantiomeric purity of the (S)-diol intermediate, comprising:

(1) combining a (S)-enantiomer enriched diol intermediate with (+)-di-p-toluoyltartaric acid in a suitable solvent or mixture of solvents;

(2) precipitating a diastereomeric salt of (+)-di-p-toluoyltartaric acid and the (S)-diol intermediate;

(3) separating the precipitate; and

(4) reacting the precipitate with a base, to obtain a substantially pure (S)-enantiomer of the diol intermediate.

The starting material, the enantiomerically enriched diol intermediate, may contain about 60% to about 80% of S-enantiomer, or about 70%, by weight.

The amount of optically active (+)-di-p-toluoyltartaric acid used may be less than, equal to, or greater than the molar equivalent of racemic diol intermediate. In embodiments, the amount of acid is in a ratio of from about 0.1 to about 1.0 mole, per mole of diol intermediate.

The diol intermediate and (+)-di-p-toluoyltartaric acid may be combined in a suitable solvent or mixture of solvents. Suitable solvents include, but are not limited to: alcohols such as methanol, ethanol, isopropyl alcohol, and n-propanol; halogenated hydrocarbons such as dichloromethane, 1,2-dichloroethane, chloroform, and carbon tetrachloride; ketones such as acetone, ethyl methyl ketone, and methyl isobutyl ketone; esters such as ethyl acetate, n-propyl acetate, n-butyl acetate, and t-butyl acetate; ethers such as diethyl ether, dimethyl ether, di-isopropylether, methyl t-butyl ether, tetrahydrofuran, and 1,4-dioxane; hydrocarbons such as toluene, xylene, n-heptane, cyclohexane, and n-hexane; nitriles such as acetonitrile and propionitrile; and any mixtures thereof.

The mixture may be stirred at suitable temperatures, such as about 25° C. to about the reflux temperature of the solvent or mixture of solvents used. The formed solid may be isolated using any techniques, such as filtration, centrifugation, or decantation.

The selective precipitation provides a precipitate wherein at least about 90%, or at least about 95%, by weight of the diol intermediate is the S-enantiomer. Optionally, the di-p-toluoyltartaric acid salt of the diol intermediate may be further treated with a suitable solvent or mixture of solvents. The treatment may involve recrystallization of salt from suitable solvent or it may involve washing of the salt using a suitable solvent. The solvents include, without limitation: lower alcohols such as methanol, ethanol, n-propanol, isopropanol, isobutanol. and t-butanol; esters such as ethyl acetate, isopropyl acetate, butyl acetate, and isobutyl acetate; halogenated hydrocarbons such as dichloromethane, dichloroethane, and chloroform; ketones such as acetone, methyl ethyl ketone, and methyl isobutyl ketone, ethers such as tetrahydrofuran, diethyl ether, diisopropyl ether, and dioxane, and any mixtures thereof.

The obtained precipitate may be further reacted with a base, in the presence of a suitable solvent, to obtain the corresponding substantially pure (S)-enantiomer of the diol intermediate.

Useful bases include sodium hydroxide, potassium hydroxide, sodium carbonate, potassium carbonate, and potassium t-butoxide. Suitable solvents include, but are not limited to: water; lower alcohols such as methanol, ethanol, n-propanol, isopropanol, n-propanol, isobutanol, and t-butanol; esters such as ethyl acetate, isopropyl acetate, butyl acetate, and isobutyl acetate; halogenated hydrocarbons such as dichloromethane, dichloroethane, and chloroform; ketones such as acetone, methyl ethyl ketone, and methyl isobutyl ketone; ethers such as tetrahydrofuran, diethyl ether, diisopropyl ether, and dioxane; and any mixtures thereof.

In embodiments, the enantiomerically enriched diol intermediate is treated with (+)-di-p-toluoyltartaric acid in ethyl acetate, and the resulting salt is subsequently treated with a base to obtain a substantially pure (S)-enantiomer of diol intermediate in high yield, such as about 80% to about 90%.

“Substantially pure” means that the material has an enantiomeric excess of at least about 90%, or at least about 95%, or at least about 98%, by weight of the desired enantiomer.

It will be understood that, if the production of a substantially pure (R)-diol intermediate is desired, the above process can readily be adapted by substituting (+)-di-p-toluoyltartaric acid for (−)-di-p-toluoyltartaric acid, with corresponding modifications to the subsequent stages.

The substantially pure (S)-enantiomer of the diol intermediate obtained may be converted to escitalopram. For example, the substantially pure (S)-enantiomer of the diol intermediate may be reacted with an acid, methanesulfonyl chloride, or p-toluenesulfonyl chloride, in a solvent or mixture of solvents, in the presence of a base. Suitable solvents include, but are not limited to: hydrocarbons such as toluene, xylene, n-heptane, cyclohexane, and n-hexane; nitriles such as acetonitrile and propionitrile; dimethylsulfoxide (DMSO); N,N-dimethylformamide (DMF); N,N-dimethylacetamide; N-methylpyrrolidione (NMP); and any mixtures thereof in various proportions without limitation. The reaction may be carried out at room temperature or, if desired, the reaction may be carried out at higher temperatures.

Escitalopram may be further converted to a pharmaceutically acceptable salt, such as the oxalate salt. The initial step for the preparation of escitalopram oxalate involves providing a solution of the free base of escitalopram in an organic solvent. Useful solvents are those in which the free base is soluble and in which the oxalate salt has limited solubility. The solution may be obtained by dissolving the free base in the solvent, or the solution may be obtained directly from a preceding synthesis step in which escitalopram free base is obtained. Non-limiting examples of organic solvents that may be used for dissolution of the free base include: esters, such as ethyl acetate and propyl acetate; C₁-C₅ ketones, such as acetone, ethyl methyl ketone, and butanone; and mixtures thereof in various proportions without limitation.

The dissolution temperatures may range from about 25° C. to about 100° C., or the boiling point of the solvent. The dissolution times may be as long as required to complete the dissolution; dissolution times from about 30 minutes to about 10 hours are frequently appropriate.

Subsequently, the escitalopram solution is combined with oxalic acid to produce the escitalopram oxalate salt. The oxalic acid may be added in the form of a solution in a suitable solvent. Examples of solvents that may be used include, but are not limited to: esters, such as ethyl acetate and propyl acetate; and C₁-C₅ ketones, for example, acetone, ethyl methyl ketone, and butanone.

Following the combination with oxalic acid, formation of oxalate salt occurs. The salt is less soluble in the solvent than the escitalopram, and therefore begins to precipitate. To enhance solid formation, the reaction mass may be allowed to stand, and/or cooled, as required to complete the solid formation. The solid escitalopram oxalate can be separated using suitable techniques, such as, for example, decantation, filtration by gravity or by suction, centrifugation, and the like. If desired, the solid can be washed with a small amount of the solvent, to remove residual mother liquor. The solid thus obtained may be dried to reduce the content of residual solvents.

Drying can be carried out using a tray dryer, vacuum oven, air oven, fluidized bed dryer, spin flash dryer, flash dryer, and the like. Drying may be carried out at temperatures from about 25° C. to about 75° C., with or without vacuum, and in the presence or absence of an inert atmosphere like nitrogen, argon, neon, and helium. Drying may be carried out for any desired time periods to achieve the desired product purity.

The obtained dry solid may contain small amounts of lumps or agglomerated material. A uniform, free flowing fine solid may be obtained by sieving, air jet milling, pulverization, or other methods known in the art. These techniques can be used to afford solid products having desired particle size distributions.

Having thus described the invention with reference to particular embodiments, those skilled in the art will appreciate modifications that do not depart from the spirit and scope of the disclosure. The following examples are set forth to further describe certain specific aspects and embodiments but are not intended to, and should not be construed to, be limiting in any way. The examples do not include detailed descriptions of conventional methods, as such methods are well known to those of ordinary skill in the art and are described in numerous publications.

### Example 1

### Preparation of (S)-4-[4-(dimethylamino)-1-(4′-fluorophenyl)-1-hydroxybutyl]-3-(hydroxymethyl)benzonitrile-p-toluoyltartrate salt

The hydrobromide salt of racemic 4-[4-(dimethylamino)-1-(4′-fluorophenyl)-1-hydroxy-butyl]-3-(hydroxymethyl)-benzonitrile (50 g) was placed into a round bottom flask. Ethyl acetate (125 mL) was added and the mixture was stirred at 25-35° C. Aqueous sodium carbonate solution (19 g in 125 mL water) was added and the mixture was stirred at about 25-35° C. for 20 minutes. The layers were separated. The aqueous layer was extracted with ethyl acetate (125 mL). The organic layers were combined and washed with water (125 mL) followed by 10% sodium chloride solution (125 mL). The organic layer was evaporated under reduced pressure. The obtained residue was mixed with isopropanol (250 mL) and the mixture was heated at 40-50° C. for 10 minutes. (−)-Di-p-toluoyltartaric acid (23.8 g) was added. The mixture was stirred at 40-50° C. for 5 hours. The formed solid was filtered and washed with isopropanol (40 mL). The filtrate was evaporated under reduced pressure. The residue was mixed with ethyl acetate (125 mL). Aqueous sodium carbonate solution (12.3 g in 125 mL water) was added and the mixture was stirred at 25-35° C. for 20 minutes. The layers were separated and the aqueous layer was extracted with ethyl acetate (125 mL). The organic layers were combined and washed with water (125 mL) followed by 10% sodium chloride solution (125 mL). The solvent was evaporated under reduced pressure and the residue was mixed with a solvent mixture containing 3% by volume methanol in ethyl acetate (400 mL). (+) Di-p-toluoyltartaric acid (15 g) was added and the mixture was stirred at 25-35° C. for 3 hours. The mixture was heated to reflux, maintained at that temperature for 2 hours, and was cooled to 25-35° C. The solid was filtered, washed with a solvent mixture containing 3% methanol in ethyl acetate (50 mL) and dried at 50° C. Yield 83.8% of single enantiomer, chiral purity 98.35%.

### Example 2

### Preparation of oxalate salt of S-(+)-1-[3-(dimethylamino)propyl]-1-(p-fluorophenyl)-5-phthalancarbonitrile

A salt of (+)-di-p-toluoyltartaric acid with (S)-4-[4-(dimethylamino)-1-(4′-fluorophenyl)-1-hydroxybutyl]-3-(hydroxymethyl)benzonitrile (10 g) was mixed with toluene (100 mL) in a round bottom flask. Sodium carbonate solution (3 g) in water (50 mL) was added. The mixture was heated to 50-60° C. for 30 minutes. The layers were separated. The aqueous layer was extracted with toluene (50 mL). The organic layers were combined and washed with water (50 mL), then with a 10% sodium chloride solution (2×50 mL). The organic layer was cooled to about 0-5° C. and DMF (10 mL) was added. Triethylamine (10.5 mL) was added. Methanesulphonyl chloride (2.2 mL) in toluene (10 mL) was added drop-wise over 30 minutes and the mixture was stirred at about 0-5° C. for 3 hours. The reaction was quenched by the addition of water (100 mL). The layers were separated and the aqueous layer was extracted with toluene (50 mL). The organic layers were combined and washed with water (2x50 mL). The solvent was evaporated under reduced pressure to obtain oil which was mixed with acetone (20 mL) and stirred for 10 minutes at 25-35° C. for dissolution. Oxalic acid (2.6 g) in acetone (20 mL) was added and the mixture was stirred at 25-35° C. for 2 hours. The mixture was cooled to 0-5° C. and stirred for 2 hours. The formed solid was filtered, washed with acetone (20 mL) and dried under reduced pressure. Yield 4.4 g, optical purity 99.17% by weight.

### Example 3

### Preparation of oxalate salt of S-(+)-1-[3-(dimethylamino)propyl]-1-(p-fluorophenyl)-5-phthalancarbonitrile

A salt of (+)-di-p-toluoyltartaric acid with (S)-4-[4-(dimethylamino)-1-(4′-fluorophenyl)-1-hydroxybutyl]-3-(hydroxymethyl)benzonitrile (10 g) was mixed with toluene (100 mL) in a round bottom flask. Sodium carbonate solution (3 g) in water (50 mL) was added. The mixture was heated to 50-60° C. for 30 minutes. The layers were separated. The aqueous layer was extracted with toluene (50 mL). The organic layers were combined and washed with water (50 mL), followed by 10% sodium chloride solution (2×50 mL). The organic layer was cooled to about 0-5° C. and DMF (10 mL) was added. Triethylamine (10.5 mL) was added. p-Toluenesulphonyl chloride (5.3 g) in toluene (10 mL) was added drop-wise over 30 minutes and the mixture was stirred at about 0-5° C. for 3 hours. The reaction was quenched by the addition of water (100 mL). The layers were separated and the aqueous layer was extracted with toluene (50 mL). The organic layers were combined and washed with water (2×50 mL). The solvent was evaporated under reduced pressure to obtain an oil, which was mixed with acetone (20 mL) and stirred for 10 minutes at 25-35° C. for dissolution. Oxalic acid (2.6 g) in acetone (20 mL) was added and the mixture was stirred at 25-35° C. for 2 hours, then cooled to 0-5° C. and stirred for 2 hours. The formed solid was filtered, washed with acetone (20 mL) and dried under reduced pressure. Yield 5.3 g, optical purity 99.3% by weight.

### Example 4

### Preparation of S-(+)-1-[3-(dimethylamino)propyl]-1-(p-fluorophenyl)-5-phthalancarbonitrile

A salt of (+)-di-p-toluoyltartaric acid with (S)-4-[4-(dimethylamino)-1-(4′-fluorophenyl)-1-hydroxybutyl]-3-(hydroxymethyl)benzonitrile (20 g), having a chiral purity of 95.63% by weight, was mixed with toluene (100 mL) in a round bottom flask. Sodium carbonate solution (6 g) in water (50 mL) was added. The mixture was heated to 50-60° C. for 30 minutes. The layers were separated. The aqueous layer was extracted with toluene (50 mL). The organic layers were combined and washed with water (100 mL), followed by 10% sodium chloride solution (2×100 mL). The organic layer was cooled to about 0-5° C. and N-methyl-2-pyrrolidone (20 mL) was added. Triethylamine (15.2 g) was added. p-Toluenesulphonyl chloride (10.6 g) in toluene (20 mL) was added drop-wise over 30 minutes and the mixture was stirred at about 0-5° C. for 3 hours. The reaction was quenched by the addition of water (100 mL). The layers were separated and the aqueous layer was extracted with toluene (50 mL). The organic layers were combined and washed with water (2×50 mL). The solvent was evaporated under reduced pressure to obtain an oil. Yield 12 g.

### Example 6

### Preparation of oxalate salt of S-(+)-1-[3-(dimethylamino)propyl]-1-(p-fluorophenyl)-5-phthalancarbonitrile

Escitalopram (6.0 g) obtained in Example 5 was mixed with ethyl acetate (20 mL) and stirred at 25-35° C. for 10 minutes. Oxalic acid (2.6 g) dissolved in ethyl acetate (20 mL) was added. The mixture was stirred at 25-30° C. for 1 hour and then stirred at 0-5° C. for 1 hour. The formed solid was filtered, washed with ethyl acetate (10 mL) and dried under vacuum. Yield 6.1 g, HPLC purity 99.83% by weight, chiral purity 95.4% by weight.

### Example 7

### Preparation of oxalate salt of S-(+)-1-[3-(dimethylamino)propyl]-1-(p-fluorophenyl)-5-phthalancarbonitrile

Escitalopram (6.0 g) obtained in Example 5 was mixed with isopropyl alcohol (40 mL) and stirred at 25-35° C. for 10 minutes. Oxalic acid (2.6 g) was added. The mixture was stirred at 25-30° C. for 1 hour and then stirred at 0-5° C. for 1 hour. The formed solid was filtered, washed with isopropyl alcohol (10 mL) and dried under vacuum. Yield 5.8 g, HPLC purity 99.91% by weight, chiral purity 94.9% by weight.

1. A process for preparing escitalopram, or a salt thereof, comprising:
a) combining racemic (±)-4-[4-(dimethylamino)-1-(4′-fluorophenyl)-1-hydroxybutyl]-3-(hydroxymethyl)benzonitrile with (−)-di-p-toluoyltartaric acid, in a solvent;
b) separating a solid phase comprising a salt of (−)-di-p-toluyltartaric acid with (R)-4-[4-(dimethylamino)-1-(4′-fluorophenyl)-1-hydroxybutyl]-3-(hydroxymethyl)benzonitrile enantiomer, and a liquid phase comprising a salt of (−)-di-p-toluoyltartaric acid with (S)-4-[4-(dimethylamino)-1-(4′-fluorophenyl)-1-hydroxy-butyl]-3-(hydroxymethyl)benzonitrile enantiomer;
c) reacting the liquid phase with a base and isolating enantiomerically enriched (S)-4-[4-(dimethylamino)-1-(4′-fluorophenyl)-1-hydroxybutyl]-3-(hydroxymethyl)benzonitrile enantiomer;
d) combining enantiomerically enriched (S)-enantiomer obtained in c) with (+)-di-p-toluoyltartaric acid, in a solvent; and
e) reacting a precipitate from d) with a base to produce (S)-4-[4-(dimethylamino)-1-(4′-fluorophenyl)-1-hydroxybutyl]-3-(hydroxymethyl)benzonitrile.

2. The process according to claim 1, wherein in a) and d) an amount of a di-p-toluoyltartaric acid independently is in a ratio from about 0.15 to about 0.95 moles, per mole of 4-[4-(dimethylamino)-1-(4′-fluorophenyl)-1-hydroxybutyl]-3-(hydroxymethyl)-benzonitrile.

3. The process according to claim 1, wherein a solvent in a) and d) independently comprises an alcohol, a ketone, an ether, a hydrocarbon, a nitrile, or any mixtures thereof.

4. The process according to claim 1, wherein a solvent in a) and d) independently comprises methanol, ethanol, isopropyl alcohol, n-propanol, dichloromethane, 1,2-dichloroethane, chloroform, carbon tetrachloride, acetone, ethyl methyl ketone, methyl isobutyl ketone, ethyl acetate, n-propyl acetate, n-butyl acetate, t-butyl acetate, diethyl ether, dimethyl ether, diisopropyl ether, methyl t-butyl ether, tetrahydrofuran, 1,4-dioxane, toluene, xylene, n-heptane, cyclohexane, n-hexane, acetonitrile, propionitrile, or any mixtures thereof.

5. The process according to claim 1, wherein reactions of a) and d) independently are conducted at temperatures about 25° C. to about the reflux temperature of a solvent or mixture of solvents used.

6. The process according to claim 1, wherein in c) and e) a base independently comprises an alkali metal or alkaline earth metal hydroxide, carbonate, or bicarbonate, or an amine.

7. The process according to claim 1, wherein in c) enantiomerically enriched 4-[4-(dimethylamino)-1-(4′-fluorophenyl)-1-hydroxybutyl]-3-(hydroxymethyl)-benzonitrile enantiomer contains at least about 60-80 percent by weight of (S)-enantiomer.

8. The process according to claim 1, further comprising purifying (S)-4-[4-(dimethylamino)-1-(4′-fluorophenyl)-1-hydroxybutyl]-3-(hydroxymethyl)benzonitrile enantiomer, by repeating d) and e).

9. The process of claim 1, further comprising converting (S)-4-[4-(dimethylamino)-1-(4′-fluorophenyl)-1-hydroxybutyl]-3-(hydroxymethyl)benzonitrile to escitalopram.

10. The process of claim 1, wherein produced (S)-4-[4-(dimethylamino)-1-(4′-fluorophenyl)-1-hydroxybutyl]-3-(hydroxymethyl)benzonitrile has enantiomeric excess at least about 95% by weight.

11. The process of claim 1, wherein produced (S)-4-[4-(dimethylamino)-1-(4′-fluorophenyl)-1-hydroxybutyl]-3-(hydroxymethyl)benzonitrile has enantiomeric excess at least about 98% by weight.

12. The process of claim 1, further comprising reacting (S)-4-[4-(dimethylamino)-1-(4′-fluorophenyl)-1-hydroxybutyl]-3-(hydroxymethyl)benzonitrile with an acid, to form a salt.

13. The process of claim 1, further comprising converting (S)-4-[4-(dimethylamino)-1-(4′-fluorophenyl)-1-hydroxybutyl]-3-(hydroxymethyl)benzonitrile to escitalopram.

14. The process of claim 1, further comprising:
f) reacting (S)-4-[4-(dimethylamino)-1-(4′-fluorophenyl)-1-hydroxybutyl]-3-(hydroxymethyl)benzonitrile with an acid, methanesulfonyl chloride, or p-toluenesulfonyl chloride, in a solvent or mixture of solvents, in the presence of a base, to form escitalopram.

15. A process for preparing escitalopram, or a salt thereof, comprising:
a) combining racemic (±)-4-[4-(dimethylamino)-1-(4′-fluorophenyl)-1-hydroxybutyl]-3-(hydroxymethyl)benzonitrile with (−)-di-p-toluoyltartaric acid, in a solvent comprising an alcohol, a ketone, an ether, a hydrocarbon, a nitrile, or any mixtures thereof;
b) separating a solid phase comprising a salt of (−)-di-p-toluyltartaric acid with (R)-4-[4-(dimethylamino)-1-(4′-fluorophenyl)-1-hydroxybutyl]-3-(hydroxymethyl)benzonitrile enantiomer, and a liquid phase comprising a salt of (−)-di-p-toluoyltartaric acid with (S)-4-[4-(dimethylamino)-1-(4′-fluorophenyl)-1-hydroxy-butyl]-3-(hydroxymethyl)benzonitrile enantiomer;
c) reacting the liquid phase with a base and isolating enantiomerically enriched (S)-4-[4-(dimethylamino)-1-(4′-fluorophenyl)-1-hydroxybutyl]-3-(hydroxymethyl)benzonitrile enantiomer;
d) combining enantiomerically enriched (S)-enantiomer obtained in c) with (+)-di-p-toluoyltartaric acid, in a solvent comprising an alcohol, a ketone, an ether, a hydrocarbon, a nitrile, or any mixtures thereof;
e) reacting a precipitate from d) with a base to produce substantially pure (S)-4-[4-(dimethylamino)-1-(4′-fluorophenyl)-1-hydroxybutyl]-3-(hydroxymethyl)benzonitrile; and
f) converting substantially pure (S)-4-[4-(dimethylamino)-1-(4′-fluorophenyl)-1-hydroxybutyl]-3-(hydroxymethyl)benzonitrile into escitalopram.

16. The process of claim 15, wherein converting in f) comprises reacting substantially pure (S)-4-[4-(dimethylamino)-1-(4′-fluorophenyl)-1-hydroxybutyl]-3-(hydroxymethyl)benzonitrile with an acid, methanesulfonyl chloride, or p-toluenesulfonyl chloride, in a solvent or mixture of solvents, in the presence of a base, to form escitalopram.

17. The process of claim 15, wherein substantially pure (S)-4-[4-(dimethylamino)-1-(4′-fluorophenyl)-1-hydroxybutyl]-3-(hydroxymethyl)benzonitrile has enantiomeric excess at least about 95% by weight.

18. The process of claim 1, wherein substantially pure (S)-4-[4-(dimethylamino)-1-(4′-fluorophenyl)-1-hydroxybutyl]-3-(hydroxymethyl)benzonitrile has enantiomeric excess at least about 98% by weight.
